(19) United States
(12) Patent Application Publication
(10) Pub. No.: US 2025/0313576 A1
(43) Pub. Date: Oct. 9, 2025

(54) COMPOUND FOR PHOTOOXYGENATION CATALYST AND MEDICINAL COMPOSITION CONTAINING SAME

(71) Applicants: The University of Tokyo, Tokyo (JP); WAKAYAMA MEDICAL UNIVERSITY, Wakayama (JP)

(72) Inventors: Motomu KANAI, Tokyo (JP); Taisuke TOMITA, Tokyo (JP); Yukiko HORI, Tokyo (JP); Takanobu SUZUKI, Tokyo (JP); Youhei SOMA, Wakayama (JP); Taka SAWAZAKI, Wakayama (JP); Makoto HIGUCHI, Chiba (JP); Hiroyuki TAKUWA, Chiba (JP)

(73) Assignees: The University of Tokyo, Tokyo (JP); WAKAYAMA MEDICAL UNIVERSITY, Wakayama (JP)

(21) Appl. No.: 18/715,293

(22) PCT Filed: Nov. 30, 2022
(86) PCT No.: PCT/JP2022/044215

§ 371 Date: Oct. 25, 2024

(30) Foreign Application Priority Data
Dec. 2, 2021 (JP) 2021-195981

(51) Int. Cl. C07D 513/04; A61K 31/4985; A61P 25/28; C07D 519/00

(52) U.S. Cl. CPC C07D 513/04; A61K 31/4985; A61P 25/28; C07D 519/00

(57) ABSTRACT

[Object] An object of the present invention is to develop an artificial catalyst capable of inhibiting aggregation of pathogenic amyloids (tau amyloids) formed by aggregation of tau proteins and to provide an agent for preventing and treating an amyloid-associated disease using the same.

[Solution] It is found that a compound having a structure in which an electron acceptor site including a specific thiazole ring is linked to an electron donor site by intramolecular conjugation is useful as a novel in vivo catalyst that selectively oxygenates tau amyloids by light irradiation and suppresses aggregation thereof. In addition, it has also been found that the compound has excellent permeability to the blood-brain barrier, and advances oxygenation of tau amyloids in the brain by light irradiation from outside the body.

### TECHNICAL FIELD

The present invention relates to a photooxygenation catalyst compound that inhibits aggregation of pathogenic amyloids, particularly tau proteins, and a pharmaceutical composition for preventing or treating a disease associated with such pathogenic amyloids.

### BACKGROUND ART

In general, proteins are responsible for vital functions by forming a specific native structure through folding. On the other hand, the proteins may be misfolded to undergo aggregation (amyloidization) into fibers rich in a B-sheet structure. Aggregates (oligomers, protofibrils, and fibers) produced in a process of the amyloidization are known to cause various dysfunctions (such diseases are collectively referred to as an “amyloid disease”). 35 types or more of proteins have been identified as causative agents of amyloid diseases. Known examples of such amyloids include amyloid β (Aβ) peptide, tau protein, α-synuclein for Parkinson's disease, amylin for diabetes mellitus, transthyretin for systemic amyloidosis, and huntingtin for Huntington's disease.

Alzheimer's disease is a progressive neurodegenerative disease that causes cognitive decline along with brain atrophy, and the number of patients is increasing year by year. This Alzheimer's disease is also a type of amyloid disease, and it is considered that neurotoxicity due to aggregates formed by amyloid β (Aβ) is involved in the onset of the Alzheimer's disease. So far, therapeutic methods targeting AR have been actively studied. So far, the inventors of the present application have developed a compound capable of reducing aggregation and toxicity of AR by a photooxygenation reaction of imparting an oxygen atom to AR (Non Patent Literatures 1 and 2 and the like).

However, since a correlation between an increase rate of AR and a degree of deterioration in cognitive function is weak, and successful applications of drug development focusing on AR to humans are currently limited, development of a new method leading to a safe and effective treatment of Alzheimer's disease is desired.

Meanwhile, in recent years, tau protein has also attracted attention as pathogenic amyloid involved in Alzheimer's disease. It is considered that neurotoxicity of amyloids formed by aggregation of tau proteins is involved in the onset of Alzheimer's disease, and a therapeutic strategy targeting tau proteins is expected to lead to complete cure of Alzheimer's disease.

However, conventional therapeutic methods targeting tau proteins have problems such as low pharmacological effects, and have not been put to practical use. An aggregate of tau proteins is greatly different from AR in that it exists in cells, a lot of the strategies that have been used for AR are not considered for membrane permeability, and therefore cannot be used, and new approaches are required. So far, studies on a catalyst for oxygenating an aggregate of tau proteins have also been reported, but since selectivity to tau proteins and membrane permeability are low, there is a problem that tau proteins cannot be oxygenated in cells and individuals (Non Patent Literature 3). Therefore, it is desired to develop a new technique that contributes to safe and effective Alzheimer's disease treatment targeting tau proteins.

### CITATION LIST

### Non Patent Literatures

Non Patent Literature 1: Taniguchi, A. et al., Nat. Chem. 2016, 8, 974-982
Non Patent Literature 2: Ni, J. et al., M. Chem 2018, 4, 807-820
Non Patent Literature 3: Suzuki, T. et al., Chem. Commun. 2019, 55, 6165-6168

### SUMMARY OF INVENTION

### Technical Problem

In view of such problems of the prior art, an object of the present invention is to develop an artificial catalyst capable of inhibiting aggregation of pathogenic amyloids (hereinafter, may be referred to as “tau amyloids”) formed by aggregation of tau proteins, and to provide an agent for preventing and treating an amyloid-associated disease using the same.

### Solution to Problem

As a result of intensive studies to solve the above problems, the present inventors have found that a compound having a structure in which an electron acceptor site including a specific thiazole ring is linked to an electron donor site by intramolecular conjugation is useful as a novel in vivo catalyst that selectively oxygenates tau amyloids by light irradiation and suppresses aggregation thereof. In addition, it has also been found that the compound has excellent permeability to the blood-brain barrier, and advances oxygenation of tau amyloids in the brain by light irradiation from outside the body. These findings have led to the completion of the present invention. Note that the term “oxygenation” is used here to broadly mean oxidation, and specifically to mean a chemical reaction that imparts and bonds oxygen atoms.

That is, in one aspect, the present invention relates to a compound suitable for photooxygenation of pathogenic amyloids, and provides:
<1> A compound represented by the following Formula (I) or a salt thereof:

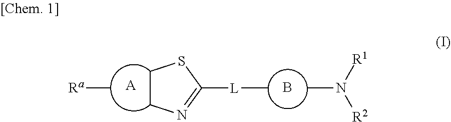

(wherein a ring A is an aromatic ring which may be substituted or a heteroaromatic ring which may be substituted; a ring B is an aromatic ring which may be substituted or a heteroaromatic ring which may be substituted; L is a conjugate spacer; R^{a} is a halogen atom or a haloalkyl group having 1 to 3 carbon atoms, and may be present at any position on the ring A; R¹ and R² may be independently the same or different, and each are independently a hydrogen atom or a substituent selected from the group consisting of an alkyl group, an alkoxy group, a hydroxyalkyl group, an ether group, a hydroxyether group, an aminoalkyl group, a thioalkyl group, and a thioether group which may be substituted, and in a case where R¹ and/or R² is an alkyl group, R¹ and/or R² may form a ring structure containing a nitrogen atom to which R¹ and/or R² is bonded and optionally an atom constituting the ring B; and the N atom to which R¹ and R² are bonded may be present at any position on the ring B);
<2> The compound according to <1>, in which the ring A is a 4- to 6-membered monocyclic aromatic ring or heteroaromatic ring;
<3> The compound according to <1> or <2>, in which the ring B is a 4- to 6-membered monocyclic aromatic ring or heteroaromatic ring, or an 8- to 12-membered bicyclic aromatic ring or heteroaromatic ring;
<4> The compound according to any one of <1> to <3>, in which R^{a} is Br or I;
<5> The compound according to any one of <1> to <4>, in which L is an alkenylene group having a conjugated double bond, an aryl group, or a combination thereof;
<6> The compound according to any one of <1> to <5>, in which R¹ and R² may be independently the same or different and each are a linear or branched alkyl group having 1 to 10 carbon atoms, a linear or branched hydroxyalkyl group having 1 to 10 carbon atoms, or a linear or branched hydroxyether group having 1 to 10 carbon atoms;
<7> The compound or the salt thereof according to <1>, in which the compound or the salt thereof is represented by the following Formula (II):

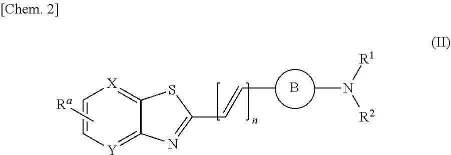

(wherein the ring B, R^{a}, R¹, and R² are as defined in <1>; X and Y may be independently the same or different, and each are a carbon atom or a nitrogen atom; and n is a natural number of 1 to 5);
<8> The compound according to <7>, in which X is a nitrogen atom, and Y is a carbon atom or a nitrogen atom;
<9> The compound or the salt thereof according to <1>, in which the compound or the salt thereof is represented by the following Formula (III):

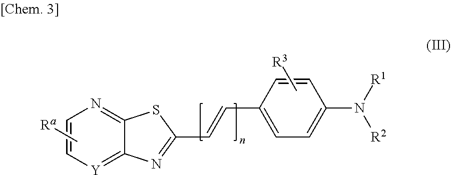

(wherein R^{a}, R¹, and R² are as defined in <1>; Y is a carbon atom or a nitrogen atom; R³s may be independently the same or different, and each are a hydrogen atom or 1 to 4 substituents selected from arbitrary substituents; and n is a natural number of 1 to 5);
<10> The compound according to <9>, in which one or two of R³s are alkoxy groups; and
<11> The compound or the salt thereof according to <9>, in which the compound or the salt thereof is represented by any one of the following Formulas (III-a) to (III-c):

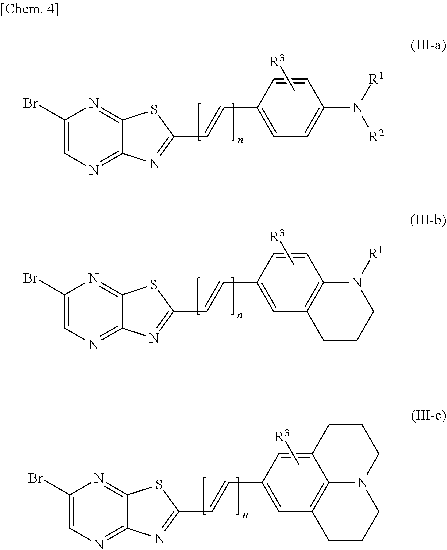

(wherein R¹, R², R³, and n are as defined in <9>).

In addition, in another aspect, the present invention relates to a pharmaceutical composition containing the compound, a method for using the compound, and the like, and more specifically, provides:
<12> A photooxygenation catalyst for pathogenic amyloids, containing the compound or the salt thereof according to any one of <1> to <11>;
<13> An aggregation inhibitor for pathogenic amyloids, containing the compound or the salt thereof according to any one of <1> to <11>;
<14> The photooxygenation catalyst according to <12> or the aggregation inhibitor according to <13>, in which the pathogenic amyloids are tau proteins;
<15> A pharmaceutical composition containing the compound or the salt thereof according to any one of <1> to <11> and a pharmaceutically acceptable carrier;
<16> The pharmaceutical composition according to <15>, in which the pharmaceutical composition is an agent for preventing or treating a disease associated with pathogenic amyloids;
<17> The pharmaceutical composition according to <16>, in which the disease associated with pathogenic amyloids is Alzheimer's disease;
<18> The pharmaceutical composition according to any one of <15> to <17>, in which the pathogenic amyloids are tau proteins;
<19> A use of the compound or the salt thereof according to any one of <1> to <11> for preparing an agent for preventing or treating a disease associated with pathogenic amyloids;
<20> A method for preventing or treating a disease associated with pathogenic amyloids, the method including administering an effective amount of the compound or the salt thereof according to any one of <1> to <11>;
<21> The method according to <20>, further including, after the administering of the compound or the salt thereof according to any one of <1> to <11>, irradiating an affected area of a patient with light from outside a body; and
<22> The method according to <20> or <21>, in which the disease associated with pathogenic amyloids is Alzheimer's disease; and
<23> The method according to any one of <20> to <22>, in which the pathogenic amyloids are tau proteins.

### Advantageous Effects of Invention

According to the present invention, it is possible to provide a novel photooxygenation catalyst compound having high catalytic activity for oxygenating pathogenic amyloids such as tau amyloids by light irradiation and excellent permeability to the blood-brain barrier. As a result, aggregation and toxicity of pathogenic amyloids in vivo (in the brain or the like) can be suppressed or reduced by a non-invasive method in which light irradiation is performed from outside the body after administration by intravenous administration or the like. Therefore, in the present invention, it is possible to prevent and treat a disease associated with pathogenic amyloids such as tau amyloids by an unconventional minimally invasive method.

### BRIEF DESCRIPTION OF DRAWINGS

FIG. 1 illustrates a UV-vis spectrum of a compound of the present invention.

FIG. 2 illustrates the results of Western blotting after performing an intracellular oxygenation reaction by compounds (PzT, OMe, Q, and J) of the present invention.

FIG. 3 illustrates the results of Western blotting after performing an intracellular oxygenation reaction by compounds (Q, C2OH, O, and O2) of the present invention.

FIG. 4 illustrates the results showing membrane permeability of compounds (Q and C2OH) of the present invention.

FIG. 5 illustrates the result showing an intracellular concentration of the compound (O) of the present invention.

FIG. 6 is a graph showing a ratio of detection intensities of methionine oxidants.

FIG. 7 is a graph showing an amino acid residual ratio before and after a photooxygenation reaction.

FIG. 8 is a graph showing a cell viability associated with addition of the compound (O) of the present invention.

FIG. 9 is a graph showing blood-brain barrier (BBB) permeability of the compound (O) of the present invention.

FIG. 10 illustrates the results of Western blotting after performing an intracellular oxygenation reaction with the compound (O) of the present invention in the brain of a model mouse.

FIG. 11 is a schematic view illustrating a procedure of an immunohistological experiment of Example 6-2.

FIG. 12 illustrates fluorescence images of a phosphorylated tau antibody (AT8) after administration of the compound (O) of the present invention in the brain of the model mouse (upper drawing: no light irradiation, lower drawing: light irradiation).

FIG. 13 is a graph showing a change in phosphorylated tau antibody (AT8) positive area after performing an intracellular oxygenation reaction with the compound (O) of the present invention in the brain of a model mouse.

FIG. 14 illustrates fluorescence images of NeuN antibody in hippocampal CA3 after administration of the compound (O) of the present invention in the brain of the model mouse (upper drawing: no light irradiation, lower drawing: light irradiation).

FIG. 15 is a graph showing a change in NeuN positive area in hippocampal CA3 after performing an intracellular oxygenation reaction with the compound (O) of the present invention in the brain of a model mouse.

### DESCRIPTION OF EMBODIMENTS

Hereinafter, embodiments of the present invention will be described. The scope of the present invention is not limited to these descriptions, and examples other than those shown below can be appropriately modified and implemented without impairing the gist of the present invention.

### 1. Definition

In the present specification, the “halogen atom” means a fluorine atom, a chlorine atom, a bromine atom, or an iodine atom.

In the present specification, the “alkyl (or alkyl group)” may be any of linear alkyl, branched alkyl, cyclic alkyl, or an aliphatic hydrocarbon group composed of a combination thereof. The number of carbon atoms in the alkyl group is not particularly limited, and is, for example, 1 to 20 carbon atoms (C₁₋₂₀), 1 to 15 carbon atoms (C₁₋₁₅), or 1 to 10 carbon atoms (C₁₋₁₀). In the present specification, the alkyl group may have one or more arbitrary substituents. For example, C₁₋₈ alkyl includes methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. Examples of the substituent include an alkoxy group, a halogen atom (may be any one of a fluorine atom, a chlorine atom, a bromine atom, or an iodine atom), an amino group, a mono- or disubstituted amino group, a substituted silyl group, and acyl, but are not limited thereto. In a case where the alkyl group has two or more substituents, these substituents may be the same or different. The same applies to alkyl moieties of other substituents including alkyl moieties (for example, an alkoxy group, an arylalkyl group, and the like).

In the present specification, the “alkylene” is a divalent group composed of a linear or branched saturated hydrocarbon, and examples thereof include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, and 2,2-di-n-propyltrimethylene.

In the present specification, the “alkenylene” is a divalent group composed of a linear or branched unsaturated hydrocarbon having at least one carbon-carbon double bond at any position, and examples thereof include ethenylene, 1-methylethenylene, 1-ethylethenylene, 1,2-dimethylethenylene, 1,2-diethylethenylene, 1-ethyl-2-methylethenylene, propenylene, 1-methyl-2-propenylene, 2-methyl-2-propenylene, 1,1-dimethyl-2-propenylene, 1,2-dimethyl-2-propenylene, 1-ethyl-2-propenylene, 2-ethyl-2-propenylene, 1,1-diethyl-2-propenylene, 1,2-diethyl-2-propenylene, 1-butenylene, 2-butenylene, 1-methyl-2-butenylene, 2-methyl-2-butenylene, 1,1-dimethyl-2-butenylene, and 1,2-dimethyl-2-butenylene.

In the present specification, the “alkynylene” is a divalent group composed of a linear or branched unsaturated hydrocarbon having at least one carbon-carbon triple bond at an arbitrary position, and examples thereof include a linear or branched divalent hydrocarbon group having 2 to 15 carbon atoms, preferably 2 to 10 carbon atoms, more preferably 2 to 6 carbon atoms, and still more preferably 2 to 4 carbon atoms. Examples thereof include acetylene, ethynylene, propynylene, butynylene, pentynylene, and hexynylene.

In the present specification, the “alkoxy” is a structure in which the alkyl group is bonded to an oxygen atom, and examples thereof include a linear alkoxy group, a branched alkoxy group, a cyclic alkoxy group, and a saturated alkoxy group composed of a combination thereof. Preferred examples thereof include a methoxy group, an ethoxy group, an n-propoxy group, an isopropoxy group, a cyclopropoxy group, an n-butoxy group, an isobutoxy group, an s-butoxy group, a t-butoxy group, a cyclobutoxy group, a cyclopropylmethoxy group, an n-pentyloxy group, a cyclopentyloxy group, a cyclopropylethyloxy group, a cyclobutylmethyloxy group, an n-hexyloxy group, a cyclohexyloxy group, a cyclopropylpropyloxy group, a cyclobutylethyloxy group, and a cyclopentylmethyloxy group.

In the present specification, the “aromatic ring” refers to a monocyclic or fused polycyclic conjugated unsaturated hydrocarbon ring structure, and the “heteroaromatic ring” refers to an aromatic ring containing one or more heteroatoms (an oxygen atom, a nitrogen atom, a sulfur atom, or the like) as ring-constituting atoms.

In the present specification, the “aryl (or aryl group)” may be either a monocyclic or fused polycyclic aromatic hydrocarbon group, and may be an aromatic heterocyclic ring containing one or more heteroatoms (for example, an oxygen atom, a nitrogen atom, a sulfur atom, or the like) as ring-constituting atoms. In this case, it is referred to as a “heteroaryl group” or a “heteroaromatic group”. Even in a case where aryl is a single ring or a fused ring, it may be bonded at all possible positions. Non-limiting examples of the monocyclic aryl include a phenyl group (Phe), a thienyl group (2- or 3-thienyl group), a pyridyl group, a furyl group, a thiazolyl group, an oxazolyl group, a pyrazolyl group, a 2-pyrazinyl group, a pyrimidinyl group, a pyrrolyl group, an imidazolyl group, a pyridazinyl group, a 3-isothiazolyl group, a 3-isoxazolyl group, a 1,2,4-oxadiazol-5-yl group, and a 1,2,4-oxadiazol-3-yl group. Non-limiting examples of the fused polycyclic aryl include a 1-naphthyl group, a 2-naphthyl group, a 1-indenyl group, a 2-indenyl group, a 2,3-dihydroinden-1-yl group, a 2,3-dihydroinden-2-yl group, a 2-anthryl group, an indazolyl group, a quinolyl group, an isoquinolyl group, a 1,2-dihydroisoquinolyl group, a 1,2,3,4-tetrahydroisoquinolyl group, an indolyl group, an isoindolyl group, a phthalazinyl group, a quinoxalinyl group, a benzofuranyl group, a 2,3-dihydrobenzofuran-1-yl group, a 2,3-dihydrobenzofuran-2-yl group, naphthyridinyl, dihydronaphthyridinyl, tetrahydronaphthyridinyl, imidazopyridinyl, pteridinyl, purinyl, quinolizinyl, indolizinyl, tetrahydroquinolizinyl, tetrahydroindolizinyl, a 2,3-dihydrobenzothiophen-1-yl group, a 2,3-dihydrobenzothiophen-2-yl group, a benzothiazolyl group, a benzimidazolyl group, a fluorenyl group, and a thioxanthenyl group. In the present specification, the aryl group may have one or more arbitrary substituents on its ring. Examples of the substituent include an alkoxy group, a halogen atom, an amino group, a mono- or disubstituted amino group, a substituted silyl group, and acyl, but are not limited thereto. In a case where the aryl group has two or more substituents, these substituents may be the same or different. The same applies to aryl moieties of other substituents including aryl moieties (for example, an aryloxy group, an arylalkyl group, and the like).

In the present specification, the “arylalkyl” represents alkyl substituted with the aryl described above. Arylalkyl may have one or more arbitrary substituents. Examples of the substituent include an alkoxy group, a halogen atom, an amino group, a mono- or disubstituted amino group, a substituted silyl group, and an acyl group, but are not limited thereto. In a case where the acyl group has two or more substituents, these substituents may be the same or different. Representative examples thereof include arylalkylbenzyl and p-methoxybenzyl.

In the present specification, the “aminoalkyl” is a group in which some or all of hydrogen atoms constituting a linear or branched alkyl group are substituted with an amino group, and examples thereof include aminomethyl, 2-aminoethyl, 1-aminoethyl, 3-aminopropyl, 4-aminobutyl, 5-aminopentyl, and 6-aminohexyl.

In the present specification, the “thioalkyl” is a group in which some or all of hydrogen atoms constituting a linear or branched alkyl group are substituted with a thiol group.

In the present specification, “alkylamino” and “arylamino” mean an amino group in which a hydrogen atom of a —NH₂ group is substituted with one or two of the alkyl or aryl. Examples thereof include methylamino, dimethylamino, ethylamino, diethylamino, ethylmethylamino, and benzylamino.

In the present specification, the “ether group” means a functional group having at least one ether bond (—O—) in an alkyl chain. Similarly, the “thioether group” means a functional group having at least one thioether bond (—S—) in an alkyl chain.

In the present specification, in a case where a certain functional group is defined as “which may be substituted”, the type of substituent, a substitution position, and the number of substituents are not particularly limited, and in a case where a certain functional group has two or more substituents, these substituents may be the same or different. Examples of the substituent include an alkyl group, an alkoxy group, a hydroxyl group, a carboxyl group, a halogen atom, a sulfo group, an amino group, an alkoxycarbonyl group, and an oxo group, but are not limited thereto. Substituents may be further present in these substituents. Examples thereof include a halogenated alkyl group, but are not limited thereto.

In the present specification, the term “ring structure” when formed by a combination of two substituents means a heterocyclic ring or carbocyclic ring, and such a ring can be saturated, unsaturated, or aromatic. Therefore, the ring structure includes cycloalkyl, cycloalkenyl, aryl, and heteroaryl as defined above.

In the present specification, a specific substituent can form a ring structure with another substituent, and in a case where such substituents are bonded to each other, those skilled in the art can understand that a specific substitution, for example, a bond to hydrogen is formed. Therefore, in a case where it is described that specific substituents together form a ring structure, those skilled in the art can understand that the ring structure can be formed by a usual chemical reaction and is easily generated. Such ring structures and a formation process thereof are all within the purview of those skilled in the art. In addition, the ring structure may have an arbitrary substituent on the ring.

### 2. Compound of the Invention

The compound of the present invention has a skeleton in which a cyclic structural site (electron acceptor site) in which a ring A and a thiazole ring are bonded and a ring B site (electron donor site) having an amino group are linked by conjugation, and is represented by the following Formula (I).

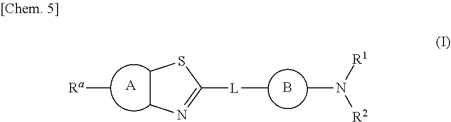

In Formula (I), the ring A is an aromatic ring which may be substituted or a heteroaromatic ring which may be substituted. The ring A is fused with a thiazole ring to form an electron acceptor site. As the heteroaromatic ring, an aromatic ring containing one or more nitrogen atoms or oxygen atoms can be typically used. The ring A can be monocyclic, bicyclic, or tricyclic, and is preferably a 4- to 6-membered monocyclic aromatic ring or heteroaromatic ring, and more preferably a 6-membered monocyclic aromatic ring or heteroaromatic ring. Preferred specific examples of the ring A include a benzene ring, a pyridine ring, a pyrazine ring, a pyrimidine ring, a pyridazine ring, a pyrrole ring, a furan ring, a pyran ring, a quinoline ring, an isoquinoline ring, a quinoxaline ring, a benzopyran ring, a naphthalene ring, and an anthracene ring. More preferably, the ring A is a benzene ring, a pyridine ring, or a pyrazine ring.

In Formula (I), the ring B is an aromatic ring which may be substituted or a heteroaromatic ring which may be substituted, and can be the same as or different from the ring A. The ring B having an amino group (NR¹(R²)) forms an electron donor site. Typically, as the ring B, an aromatic ring containing one or more nitrogen atoms or oxygen atoms can be used. The ring B can be monocyclic, bicyclic, or tricyclic, and is preferably a 4- to 6-membered monocyclic aromatic ring or heteroaromatic ring or an 8- to 12-membered bicyclic aromatic ring or heteroaromatic ring. More preferably, the ring B is a 6-membered monocyclic aromatic ring or heteroaromatic ring. Preferred specific examples of the ring B include a benzene ring, a pyridine ring, a pyrazine ring, a pyrimidine ring, a pyridazine ring, a pyrrole ring, a furan ring, a pyran ring, a quinoline ring, an isoquinoline ring, a quinoxaline ring, a benzopyran ring, a naphthalene ring, an anthracene ring, a thiophene ring, a tetrahydroquinoline ring, and a tetrahydroisoquinoline ring. More preferably, the ring B is a benzene ring or a naphthalene ring.

In Formula (I), L is a conjugate spacer, and means a divalent group capable of linking between a cyclic structure site in which a ring A and a thiazole ring are bonded and a ring B site having an amino group while maintaining a conjugated system. The conjugate spacer may have a structure having a conjugated double bond, and examples thereof include an alkenylene group, an aryl group, and a combination thereof. Note that as long as a conjugated system can be maintained, the conjugate spacer may contain any heteroatom such as thiophene, and may further have a substituent such as an alkyl group, an alkoxy group, an aryl group, a halogen atom, an alkenyl group, an alkynyl group, a carbonyl group, a cyano group, a nitro group, a phosphoryl group, or a sulfonyl group at a substitutable position. L is preferably an alkenylene group or an aryl group, and more preferably any alkenylene group having 2 to 10 carbon atoms, a phenylene group, or a combination thereof.

In Formula (I), R^{a} is a group capable of causing a heavy atom effect, and is an electron withdrawing group. Specifically, R^{a} is a halogen atom or a haloalkyl group having 1 to 3 carbon atoms. R³ is preferably a bromine atom (Br) or an iodine atom (I). R^{a} can be present at any position of the ring A.

In Formula (I), R¹ and R² may be independently the same or different, and can be each independently a hydrogen atom or a substituent selected from the group consisting of an alkyl group, an alkoxy group, a hydroxyalkyl group, an ether group, a hydroxyether group, an aminoalkyl group, a thioalkyl group, and a thioether group which may be substituted. Preferably, R¹ and R² may be independently the same or different and may be a linear or branched alkyl group having 1 to 10 carbon atoms, a linear or branched hydroxyalkyl group having 1 to 10 carbon atoms, or a linear or branched hydroxyether group having 1 to 10 carbon atoms, and each of these substituents may be optionally substituted. More preferably, both R¹ and R² are linear or branched alkyl groups having 1 to 10 carbon atoms which may be the same or different. Note that the N atom to which R¹ and R² are bonded may be present at any position on the ring B.

For example, typical examples of the hydroxyalkyl group and the hydroxyether group that can be used for R¹ and R² include substituents having the following structures. However, the present invention is not limited thereto.

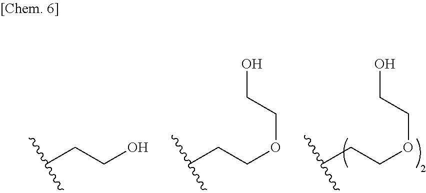

In a preferred aspect, in a case where R¹ and/or R² is an alkyl group, a ring structure containing a nitrogen atom to which R¹ and/or R² is bonded and optionally an atom constituting the ring B may be formed. For example, either R¹ or R² can form a first ring structure containing a nitrogen atom to which R¹ or R² is bonded and optionally an atom constituting the ring B. Alternatively, R¹ can form a first ring structure containing a nitrogen atom to which R¹ is bonded and optionally an atom constituting the ring B, and R² can form a second ring structure containing a nitrogen atom to which R² is bonded and optionally an atom constituting the ring B. In this case, as shown in Formula (III-c) described below, the first and second ring structures may have structures connected to each other. Such a ring structure can be, for example, a 4- to 10-membered ring, and preferably a 4- to 6-membered ring.

As a more specific aspect, in a case where the ring A is a 6-membered monocyclic aromatic ring or heteroaromatic ring, and the conjugate spacer L is an alkenylene group having a conjugated double bond, the compound of the present invention has a structure represented by the following Formula (II).

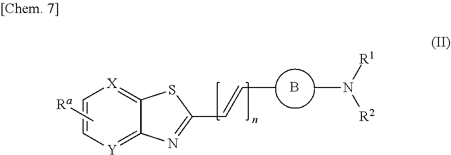

In Formula (II), the ring B, R^{a}, R¹, and R² are as defined in Formula (I). Note that R^{a} is an ortho position or a meta position with respect to Y, and is preferably a meta position.

In Formula (II), X and Y may be independently the same or different, and each are a carbon atom or a nitrogen atom. Preferably, at least one of X and Y is a nitrogen atom. More preferably, X is a nitrogen atom, and Y is a carbon atom or a nitrogen atom. Most preferably, both X and Y are nitrogen atoms (in this case, the ring A is a pyrazine ring).

In Formula (II), n is a natural number of 1 to 5, and preferably a natural number of 2 to 5.

As still another specific aspect, in Formula (II), in a case where the ring B is a 6-membered monocyclic aromatic ring, that is, a benzene ring which may have a substituent, the compound of the present invention has a structure represented by the following Formula (III).

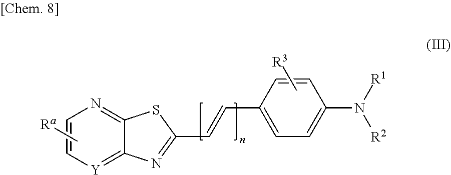

In Formula (III), R^{a}, R¹, and R² are as defined in Formula (I). Note that R^{a} is an ortho position or a meta position with respect to Y, and is preferably a meta position.

In Formula (III), Y is a carbon atom or a nitrogen atom. R³s may be independently the same or different, and each are a hydrogen atom or 1 to 4 substituents selected from arbitrary substituents. Examples of the arbitrary substituents include an alkyl group, an alkoxy group, a hydroxyalkyl group, an ether group, a hydroxyether group, an aminoalkyl group, a thioalkyl group, a thioether group, and a halogenated alkyl group. These substituents can be, for example, 1 to 10 carbon atoms, and preferably 1 to 5 carbon atoms. In a specific aspect, one or two of R³s can be alkoxy groups having 1 to 5 carbon atoms.

In Formula (III), n is a natural number of 1 to 5, and preferably a natural number of 2 to 5.

In a further specific aspect, the compound of the present invention has a structure represented by any one of the following Formulas (III-a) to (III-c).

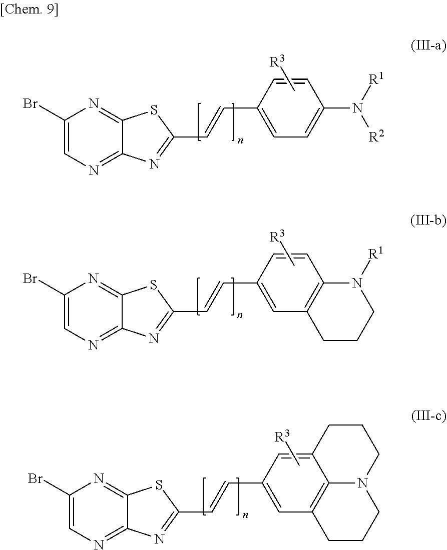

In the following Formulas (III-a) to (III-c), R¹, R², R³, and n are as defined in Formula (III).

The compound of the present invention may be present as a salt. Examples of such a salt include a base addition salt, an acid addition salt, and an amino acid salt. Examples of the base addition salt include metal salts such as a sodium salt, a potassium salt, a calcium salt, and a magnesium salt, an ammonium salt, and organic amine salts such as a triethylamine salt, a piperidine salt, and a morpholine salt, and examples of the acid addition salt include mineral acid salts such as a hydrochloride, a sulfate, and a nitrate, and organic acid salts such as a carboxylate, a methanesulfonate, a paratoluenesulfonate, a citrate, and an oxalate. Examples of the amino acid salt include a glycine salt. However, the present invention is not limited to these salts.

The compound of the present invention may have one or two or more asymmetric carbon atoms depending on the type of substituent, and a stereoisomer such as an optical isomer or a diastereoisomer may be present. A stereoisomer in pure form, any mixture of stereoisomers, a racemate, and the like are all encompassed within the scope of the present invention.

In addition, the compound or the salt thereof of the present invention may also exist as a hydrate or a solvate, and all of these substances are encompassed within the scope of the present invention. The type of solvent for forming the solvate is not particularly limited, and examples thereof include solvents such as water, ethanol, acetone, and isopropanol.

In Examples of the present specification, a method for producing a representative compound included in the compound of the present invention is specifically shown, and therefore, those skilled in the art can easily produce any compound included in Formula (I) by referring to the disclosure of the present specification and appropriately selecting a starting material, a reagent, reaction conditions, and the like as necessary based on common technical knowledge in the art. Typically, as shown in Examples, the compound of the present invention can be obtained by condensing a thiazole ring with an amine compound having a skeleton corresponding to the ring A of Formula (I) as a starting material, and then appropriately introducing a site corresponding to an L-ring B.

### 2. Pharmaceutical Composition and the Like Containing Compound of the Present Invention

The compound of the present invention can catalyze an oxygenation reaction of pathogenic amyloids, particularly tau amyloids formed by aggregation of tau proteins. The oxygenation reaction proceeds by bringing the compound of the present invention into an excited state by light irradiation to generate singlet oxygen, and adding an oxygen atom to an amino acid residue in amyloid to oxidize the amino acid residue. Therefore, it is possible to suppress or reduce aggregation of pathogenic amyloids. The compound of the present invention has excellent oxygenation activity to tau amyloids, which are formed by aggregation of tau proteins.

Therefore, in another aspect, the present invention relates to a photooxygenation catalyst for pathogenic amyloids or an aggregation inhibitor for pathogenic amyloids containing the compound or the salt thereof. Furthermore, the present invention also relates to a pharmaceutical composition containing the compound or the salt thereof and a pharmaceutically acceptable carrier. The pharmaceutical composition can be an agent for preventing or treating a disease associated with pathogenic amyloids.

The “pathogenic amyloids” include tau proteins, and amyloids such as amyloid β (Aβ) peptide, amylin, transthyretin, α-synuclein, and huntingtin, which are known to be involved in Alzheimer's disease, Parkinson's disease, diabetes mellitus, Huntington's disease, and systemic amyloidosis in animals including humans. Preferably, the pathogenic amyloid is an amyloid formed by aggregation of tau proteins.

For example, in a case where tau amyloids are oxidized by the compound of the present invention, it is sufficient that one or more amino acid residues among the amino acid residues constituting the tau proteins are oxidized, and it is preferable that one or more amino acid residues selected from His and Met are oxidized. The oxidation is more preferably in a form in which a hydroxy group or an oxo group (oxide) is added to each amino acid residue. In the case of His, an oxidant of the amino acid residue is presumed to have a structure in which an imidazole ring of a histidine residue is oxidized, that is, a dehydroimidazolone ring or a hydroxyimidazolone ring. In addition, in the case of Met, it is presumed that oxygen is added to a sulfur atom in a methionine residue.

The compound of the present invention preferably has a maximum absorption wavelength (Amax) in a range of 450 to 900 nm, and preferably can be brought into an excited state at the wavelength. From the viewpoint of avoiding cytotoxicity due to light irradiation, the compound of the present invention desirably has an absorption band in a region of 595 nm or more. The absorption band is included in such a long wavelength, such that excitation can be performed by long-wavelength light having high biological permeability.

In addition, the compound of the present invention preferably has a molecular weight of 300 to 550. The compound of the present invention has such a relatively small molecular weight, such that excellent permeability to the blood-brain barrier can be exhibited.

A pharmaceutical composition containing the compound or the salt thereof of the present invention can be prepared by a preparation method of various preparations by selecting an appropriate preparation according to an administration method and using a pharmaceutically acceptable carrier. Examples of a dosage form of the pharmaceutical composition containing the compound of the present invention as a main agent include tablets, powders, granules, capsules, liquids, syrups, elixirs, and oily or aqueous suspensions as oral preparations.

As an injection, a stabilizer, a preservative, or a solubilizing agent may be used in the preparation, and a solution containing these auxiliary agents may be stored in a container and then freeze-dried to be used as a solid preparation. In addition, a single dose may be stored in one container, or a large dose may be stored in one container.

In addition, examples of an external preparation include liquids, suspensions, emulsions, ointments, gels, creams, lotions, sprays, and patches.

The solid preparation contains a pharmaceutically acceptable additive together with the compound of the present invention, for example, fillers, bulking agents, binders, disintegrants, dissolution accelerators, wetting agents, lubricants, and the like can be selected, mixed and formulated as needed. Examples of a liquid preparation include solutions, suspensions, and emulsions, and the additive may include suspending agents, emulsifiers, and the like.

In a case where the compound of the present invention is used as a drug for a human body, a dose is preferably in a range of 1 mg to 1 g, and preferably 1 mg to 300 mg per day for an adult.

In a further aspect, the present invention also relates to a method for preventing or treating a disease associated with pathogenic amyloids, the method including administering an effective amount of the compound or the salt thereof. The method further includes, after the administering of the compound or the salt thereof, irradiating an affected area of a patient with light from outside a body. As described above, since the compound of the present invention can be excited by long wavelength light that is highly permeable to a living body, aggregation and toxicity of pathogenic amyloids in vivo (in the brain or the like) can be suppressed or reduced by a non-invasive method in which light irradiation is performed from outside the body of the patient after administration by intravenous administration or the like.

Specifically, the compound or the salt thereof of the present invention may be introduced into a living body or a cell, and irradiated with light at the time when the compound moves to a target site. Examples of the means for administration into the living body include intramuscular injection, intravenous injection, local administration, and oral administration.

Examples of the disease associated with pathogenic amyloids include Alzheimer's disease, Parkinson's disease, diabetes mellitus, Huntington's disease, and systemic amyloidosis in animals including humans. Typically, a disease associated with pathogenic amyloids is Alzheimer's disease.

### EXAMPLES

Hereinafter, the present invention will be described in more detail with reference to Examples, but the present invention is not limited to these Examples.

### 1. Synthesis of Compounds of the Invention

### 1-1. Synthesis of Compound PT

A compound PT of the present invention was synthesized by the following synthesis scheme. 2,6-Dibromo-3-aminopyridine was used as a raw material, and first, an amino group was acetylated with bromoacetyl bromide to obtain a compound 1. The compound 1 can be purified by thermal recrystallization of ethanol. Subsequently, the amide bond of the compound 1 was converted into thioamide using P₄S₁₀. However, intramolecular cyclization proceeded subsequently, and actually 2 was isolated. A phosphonate derivative 3 was obtained by Michaelis-Arbuzov reaction. Finally, Horner-Wadsworth-Emmons reaction was utilized to condense with 4-dimethylaminocinnamaldehyde to obtain the compound PT.

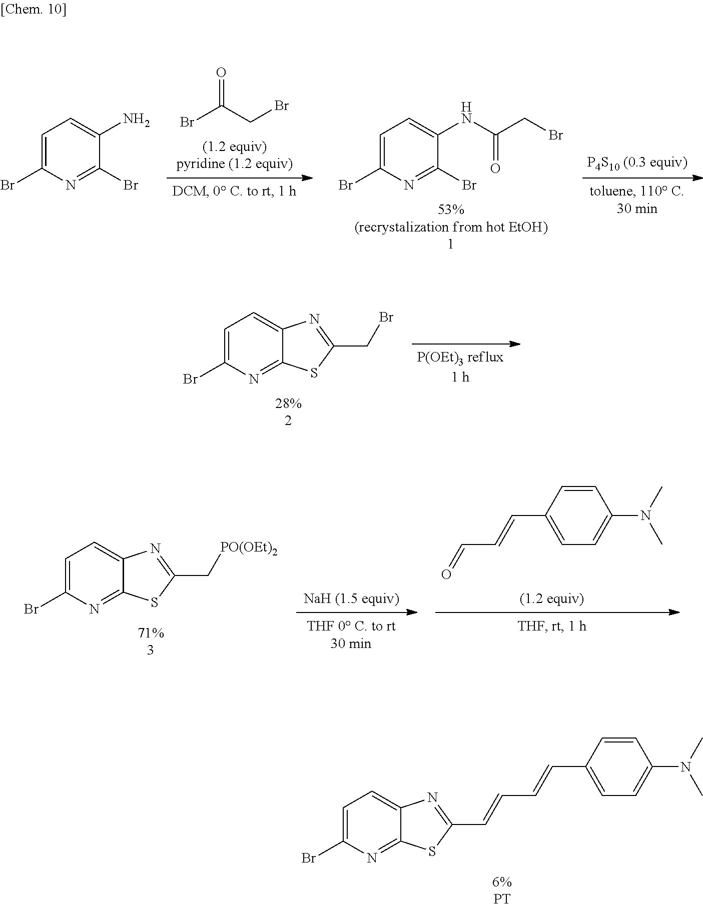

Specific synthesis conditions in each step are as follows. The following aldehydes “Ald1” and “Ald2” used were synthesized according to the existing literatures.

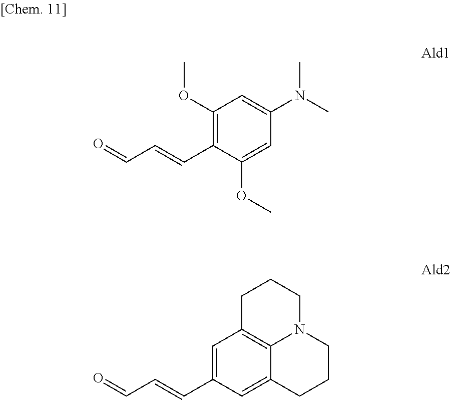 [SMILES: COc1cc(N(C)C)cc(OC)c1/C=C/C=O ; O=C/C=C/c1cc2c3c(c1)CCCN3CCC2]

### Synthesis of Compound 1 (2-Bromo-N-(2,6-dibromopyridin-3-yl) acetamide)

To DCM solution of 3-amino-2,6-bromopyridine (1.01 g, 4.0 mmol), pyridine (0.39 mL, 4.8 mmol) was added dropwise at 0° C., and subsequently, bromoacetyl bromide (0.42 mL, 4.8 mmol) was added at 0° C. After the mixture was stirred at room temperature for 30 minutes, the mixture was washed with water, dried with Na₂SO₄, filtered, and concentrated under reduced pressure. The residue was purified by silica gel column chromatography (hexane/EtOAc=1:1, Rf=0.5) to obtain a white solid compound 1 (1.25 g, 85%).

¹H NMR (392 MHz, CDCl₃): δ 8.80 (brs, 1H), 8.55 (d, J=8.5 Hz, 1H), 7.46 (d, J=8.5 Hz, 1H), 4.07 (s, 2H)

¹³C{¹H}NMR (98.5 MHz, CDCl₃): δ 164.2, 134.3, 132.7, 131.7, 130.3, 127.9, 29.3

HRMS (ESI+): m/z calcd for C₇H5N₂O₁Br₃Na⁺[M+Na]⁺:392.7850 found 392.7840

### Synthesis of Compound 2 (5-Bromo-2-(bromomethyl)thiazolo[5,4-b]pyridine)

A mixture of the compound 1 (369.8 mg, 1.00 mmol) and P₄S₁₀ (133.4 mg, 0.30 mmol) in toluene (1.0 mL) was stirred at 100° C. for 30 minutes. After the mixture was filtered at room temperature, the filtrate was concentrated and purified by silica gel column chromatography (hexane/EtOAc=4:1, Rf=0.5) to obtain a white solid compound 2 (63.3 mg, 21%).

¹H NMR (392 MHz, CDCl₃): δ 8.09 (d, J=8.5 Hz, 1H), 7.61 (d, J=8.5 Hz, 1H), 4.76 (s, 2H)

¹³C{¹H}NMR (98.5 MHz, CDCl₃): δ 168.0, 158.9, 145.3, 139.3, 132.6, 126.4, 27.2

HRMS (ESI⁺):m/z calcd for C₇H4N₂SBr₂Na⁺[M+Na]⁺328.8360 found 328.8359

### Synthesis of Compound 3 (Diethyl((5-bromothiazolo[5,4-b]pyridin-2-yl)methyl)phosphonate)

A mixture of the compound 2 (152.5 mg, 0.4986 mmol) and triethyl phosphite (0.5 mL) was stirred at 150° C. for 2 hours. After Ar was flowed at 150° C. for 15 minutes, the mixture was purified by silica gel column chromatography with AcOEt to obtain a colorless oily compound 3 (128.7 mg, 71%).

¹H NMR (500 MHz, CDCl₃): δ 8.05 (d, J=8.6 Hz, 1H), 7.55 (d, J=8.0 Hz, 1H), 4.12-4.18 (m, 4H), 3.71 (s, 1H), 3.69 (d, J=21.8 Hz, 2H), 1.31 (t, J=7.4 Hz, 6H)

¹³C{¹H}NMR (126 MHz, CDCl₃): δ 162.8 (d, J=9.6 Hz), 158.6, 145.1 (d, J=2.4 Hz), 138.3, 131.7, 125.5, 62.9 (d, J=7.2 Hz), 33.8 (d, J=138 Hz), 16.2 (d, J=6.0 Hz)

HRMS (ESI⁺): m/z calcd for C₁₃H₁₉ON₂SBrPNa⁺[M+Na]⁺386.9544 found 386.9537

### Synthesis of Compound PT (4-((1E,3E)-4-(5-bromothiazolo[5,4-b]pyridin-2-yl)butadien-1,3-dien-1-yl)-N,N-dimethylaniline)

Sodium hydride (10.4 mg, 0.26 mmol) was added to THF solution (63.2 mg, 0.17 mmol) of the compound 3 at 0° C. After the mixture was stirred at room temperature for 30 minutes, 4-dimethylaminocinnamaldehyde (45.6 mg, 0.26 mmol) was added. After the mixture was stirred at room temperature for 1 hour, the mixture was concentrated under reduced pressure. The residue was purified by preparative HPLC (RT=46.0 minutes, YMC-Triart PFP, 20 mmID×250 mm, linear gradient; with 0 to 100% acetonitrile in 0.1% aqueous TFA solution for 40 minutes or longer, then with 100% acetonitrile at a flow rate of 5 mL/min for 20 minutes) to obtain an orange solid compound PT (3.6 mg, 6%). Note that, since the compound PT was sensitive to light, the entire synthesis step was performed with light shielding.

### 1-2. Synthesis of Compound PzT

A compound PzT of the present invention was synthesized by the following synthesis scheme. A compound 4 was obtained by first acetylating 2-amino-3,5-dibromopyrazine as a starting material. The compound 4 can be crystallized from THF/hexane. Subsequently, the amide bond was converted into a thioamide bond using P₄S₁₀, and intramolecular cyclization was performed to obtain a compound 5. Finally, a compound PzT was obtained by Knoevenagel condensation with 4-dimethylaminocinnamyl aldehyde.

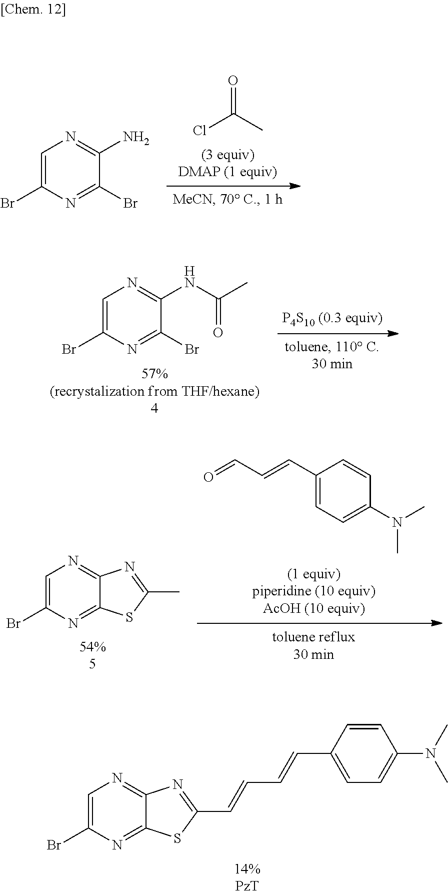

### Synthesis of Compound 4 (N-(3,5-Dibromopyrazin-2-yl) acetamide)

To a mixture of 2-amino-3,5-dibromopyrazine (10.0 g, 39.5 mmol) and DMAP (4.83 g, 39.5 mmol) in MeCN (30 mL), acetyl chloride (5.6 mL, 78.5 mmol) was added at room temperature. After the mixture was stirred at 70° C. for 2 hours, the mixture was concentrated under reduced pressure. The residue was dissolved in EtOAc. The organic layer was washed with water and the saturated aqueous NH₄Cl solution was dried with Na₂SO₄, filtered, concentrated, and purified by recrystallization from THF/hexane to obtain a needle-shaped crystalline compound 4 (6.60 g, 57%).

¹H NMR (500 MHz, DMSO-d6), inseparable mixture of rotamers (1:0.6 ratio);

major: δ 10.48 (brs, 1H), 8.74 (s, 1H), 2.10 (s, 3H)

minor: δ 10.53 (brs, 1H), 8.72 (s, 1H), 2.11 (s, 3H)

¹³C{¹H}NMR (126 Mhz, CDCl₃): δ 168.8, 168.7, 146.6, 144.9, 144.2, 144.1, 141.9, 135.3, 133.3, 132.7, 23.11, 23.09

HRMS (ESI⁺):m/z calcd for C₆H₆ON₃Br₂Na⁺[M+Na]⁺315.8697 found 315.8694

### Synthesis of Compound 5 (6-Bromo-2-methylthiazolo[4,5-b]pyrazine)

A mixture of the compound 4 (659.5 mg, 2.25 mmol) and P₄S₁₀ (300.3 mg, 0.68 mmol) in toluene (10 mL) was stirred at 110° C. for 1 hour. After the mixture was filtered at room temperature, the filtrate was concentrated and purified by silica gel column chromatography (hexane/EtOAc=1/1, Rf=0.5) to obtain a white solid compound 5 (380.7 mg, 74%).

¹H NMR (500 MHz, CDCl₃): δ 8.71 (s, 1H), 2.93 (s. 3H)

¹³C{¹H}NMR (126 MHz, CDCl₃): δ 173.2, 156.7, 152.6, 144.7, 135.8, 22.1

HRMS (ESI⁺):m/z calcd for CsH₂N₃SBrNa⁺[M+Na]⁺251.9207 found 251.9211

### Synthesis of Compound PzT (4-((1E,3E)-4-(6-Bromothiazolo[4,5-b]pyrazin-2-yl)butadien-1,3-dien-1-yl)-N,N-dimethylaniline)

To a mixture of the compound 5 (45.8 mg, 0.20 mmol) and dimethylaminocinnamaldehyde (35.0 mg, 0.20 mmol) in toluene (2 mL), AcOH (114.5 μL) and piperidine (197.6 μL) were added. After the mixture was heated at reflux for 30 minutes, the solvent was removed in vacuo. The residue was purified by preparative HPLC (RT=43.2 minutes, YMC-Triart C18, 20 mmID×250 mm, linear gradient; with 0 to 100% acetonitrile in 0.1% aqueous TFA solution at a flow rate of 8 mL/min for 40 minutes) to obtain a red solid compound PzT (10.7 mg, 14%).

¹H NMR (500 MHz, CDCl₃): δ 8.64 (s, 1H), 7.54 (dd, J=10.9, 14.9 Hz, 1H), 7.42 (d, J=8.6 Hz, 2H), 6.95 (d, J=15.5 Hz, 1H), 6.87-6.82 (m, 2H), 6.73 (d, J=8.6 Hz, 2H), 3.04 (s, 6H)

¹³C{¹H}NMR (126 MHz, CDCl₃): δ 171.5, 157.5, 151.7, 150.7, 144.4, 142.8, 142.0, 134.5, 129.6, 129.0, 122.1, 121.6, 112.0, 40.1

HRMS (ESI⁺):m/z calcd for C₁₇H₁₅N₄SBrNa⁺[M+Na]⁺409.0098 found 409.0094.

### 1-3. Synthesis of Compounds OMe, Q, and J

Next, for the purpose of obtaining an absorption band on a longer wavelength, three kinds of compounds (OMe, Q, and J) in which an aniline moiety as a donor site in the compound PzT was changed to the following structure were synthesized. In order to narrow a HOMO-LUMO gap by introducing a functional group having a higher LUMO (high electron donating property), introduction of an electron donating group (methoxy group) or free rotation of a nitrogen atom is suppressed, and an unshared electron pair on the N atom is made more likely to flow into a n linker.

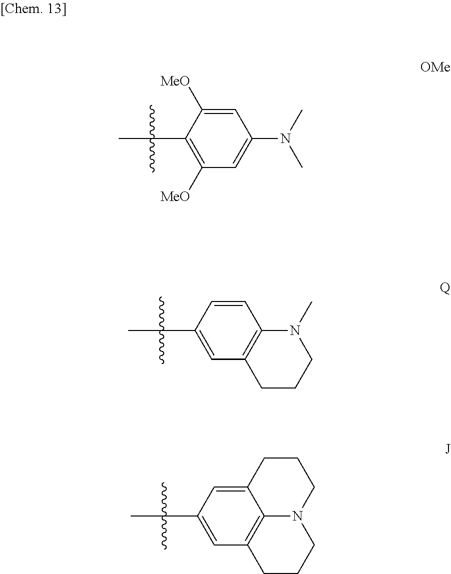 [SMILES: *c1c(OC)cc(N(C)C)cc1OC ; *c1ccc2c(c1)CCCN2C ; *c1cc2c3c(c1)CCCN3CCC2]

### 1-3a. Synthesis of Compound OMe

As shown in the following synthesis scheme, the compound OMe of the present invention was synthesized by Knoevenagel condensation of the compound 5 with 4-dimethylamino-2,6-dimethoxycinnamyl aldehyde.

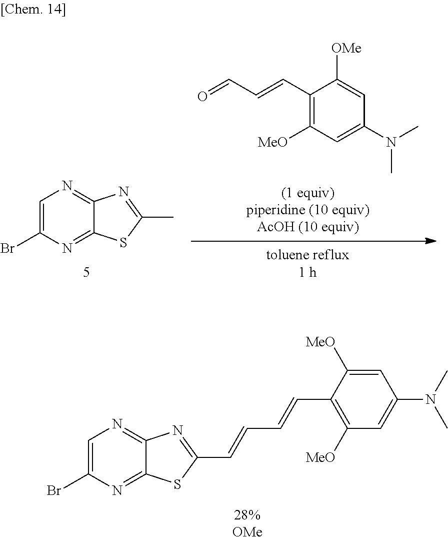 [SMILES: COc1cc(N(C)C)cc(OC)c1/C=C/C=O ; Cc1nc2ncc(Br)nc2s1 ; COc1cc(N(C)C)cc(OC)c1/C=C/C=C/c1nc2ncc(Br)nc2s1]

### Synthesis of Compound OMe (4-((1E,3E)-4-(6-Bromothiazolo[4,5-b]pyrazin-2-yl)butadien-1,3-dien-1-yl)-3,5-dimethoxy-N,N-dimethylaniline)

To a solution of the Ald1 (22.0 mg, 0.093 mmol), the compound 5 (21.4 mg, 0.093 mmol), and preactivated MS3A in toluene (1.0 mL), AcOH (53.5 μL) and piperidine (92.3 μL) were added. After the mixture was refluxed for 20 minutes, the solvent was removed in vacuo. The residue was purified by preparative HPLC (RT=91.3 minutes, YMC-Triart PEP, 20 mmID×250 mm, linear gradient; with 20 to 100% acetonitrile in 0.1% aqueous HCHO solution at a flow rate of 5 mL/min for 100 minutes). A red solid compound OMe (2.1 mg, 5%) was obtained.

¹H NMR (500 MHz, CDCl₃): δ 8.59 (s, 1H), 7.53 (dd, J=10.9, 14.9 Hz, 1H), 7.41 (d, J=15.5 Hz, 1H), XX, 6.78 (d, J=14.9 Hz, 1H), 5.82 (s, 2H), 3.91 (s, 6H), 3.06 (d, 6H)

HRMS (ESI⁺):m/z calcd for C₁₈H₁₉O₂N₄SBrNa⁺[M+Na]⁺469.0310 found 469.0304

### 1-3b. Synthesis of Compound Q

A compound Q was synthesized by the following synthesis scheme. 1,2,3,4-Tetrahydroquinoline was reductively aminated and methylated. The crude product was formylated by Vilsmeier reaction and then isolated since the product was difficult to isolate due to too high volatility. A compound 6 was obtained with a yield of 46% by the above 2 steps. Next, a compound 7 was obtained by Horner-Wadsworth-Emmons reaction and further deprotection. Finally, a compound Q was obtained by Knoevenagel condensation with the compounds 5 and 7.

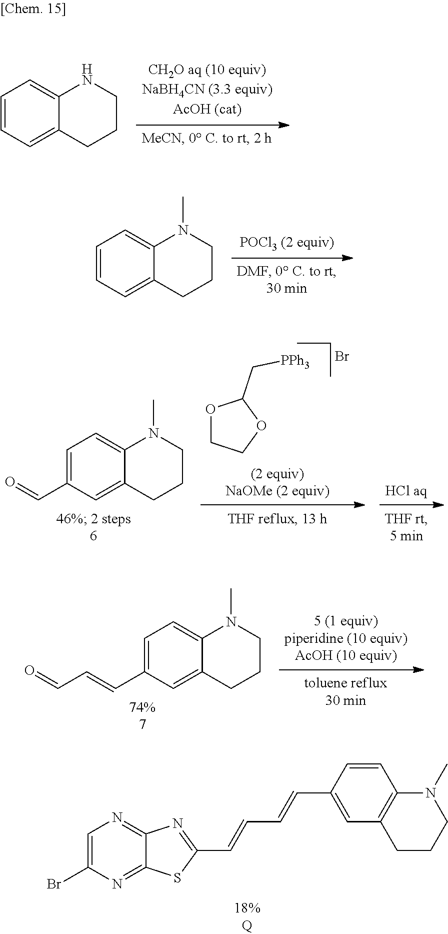

### Synthesis of Compound 6 (1-Methyl-1,2,3,4-tetrahydroquinoline-6-carbaldehyde)

To MeCN solution of 1,2,3,4-tetrahydroquinoline (1.3 mL, 10.35 mmol), formalin (8.2 mL, 10 equivalent), sodium cyanoborohydride (2.2 g, 3.3 equivalent), and acetic acid (160 μL, cat.) were added. After the mixture was stirred at room temperature for 1 hour, the mixture was diluted with DCM. The organic layer was washed with H₂O, dried with Na₂SO₄, filtered, and concentrated to obtain 1-methyl-1,2,3,4-tetrahydroquinoline as a crude material. DMF (5 mL) was added to the crude material, and the solution was cooled to 0° C. Phosphoryl chloride (1.9 mL, 20.38 mmol) was added to the solution. After the mixture was stirred at room temperature for 30 minutes, the reaction was quenched by the addition of water, and extraction was performed with DCM. The combined organic layers were washed with brine, dried with Na₂SO₄, filtered, and concentrated. The residue was purified by silica gel column chromatography (hexane/EtOAc=4/1, Rf=0.3) to obtain a colorless oily compound 6 (835.8 mg, 46%).

¹H NMR (392 MHz, CDCl₃): δ 9.58 (s, 1H), 7.47 (d, J=8.5 Hz, 1H), 7.36 (s, 1H), 6.47 (d, J=8.5 Hz, 1H), 3.29 (t, J=5.8 Hz, 2H), 2.91 (s. 3H), 2.68 (t, J=6.3 Hz, 2H), 1.87 (tt, J=6.3, 5.8 Hz, 2H)

¹³C{¹H}NMR (CDCl₃): δ 198.95, 151.09, 131.17, 129.62, 124.58, 121.77, 109.17, 50.95, 38.60, 27.48, 21.37

HRMS (ESI⁺):m/z calcd for C₁₁H₁₃ONNa⁺[M+Na]⁺198.0895 found 198.0895

### Synthesis of Compound 7 ((E)-3-(1-Methyl-1,2,3,4-tetrahydroquinolin-6-yl)acrylaldehyde)

To a solution of the compound 6 (756.3 mg, 4.32 mmol) and (1,3-dioxolane-2-yl)methyltriphenylphosphonium bromide (3.7 g, 8.62 mmol) in THF, sodium methoxide (466.7 mg, 4.32 mmol) was added. After the mixture was refluxed for 13 hours, the mixture was diluted with EtOAc. The organic layer was washed with H₂O, dried with Na₂SO₄, and filtered. To the residue, THF and a 1 N aqueous HCl solution (5 mL each) were added, and the mixture was stirred at room temperature for 5 minutes. The solution was diluted with EtOAc, the organic layer was washed with H₂O, dried with Na₂SO₄, filtered, and concentrated. The residue was purified by silica gel column chromatography (hexane/EtOAc=4/1, Rf=0.4) to obtain a yellow solid compound 7 (639.6 mg, 74%).

¹H NMR (500 MHz, CDCl₃): δ 9.55 (d, J=8.0 Hz, 1H), 7.44 (d, J=15.5 Hz, 1H), 7.37 (dd, J=8.0, 13.7 Hz, 1H), 7.27 (s, 1H), 6.61 (d, J=8.6 Hz, 1H), 6.45-6.50 (m, 1H), 3.37 (t, J=5.7 Hz, 2H), 2.98 (s, 3H), 2.76 (t, J=6.3 Hz, 2H), 1.92-1.97 (m, 2H)

¹³C{¹H}NMR (126 MHz, CDCl₃): δ 193.61, 154.14, 149.02, 129.29, 129.00, 123.07, 122.35, 121.32, 110.02, 50.99, 38.61, 27.61, 21.65

HRMS (ESI⁺):m/z calcd for C₁₃H₁₅ONNa⁺[M+Na]⁺224.1051. Found 224.1043

### Synthesis of Compound Q (6-Bromo-2-((1E,3E)-4-(1-methyl-1,2,3,4-tetrahydroquinolin-6-yl)butadiene-1,3-dien-1-yl)thiazolo[4,5-b]pyrazine)

To a solution of the compound 5 (45.8 mg, 0.20 mmol) and the compound 7 (40.2 mg, 0.20 mmol) in toluene (2 mL), AcOH (114.5 μL) and piperidine (197.6 μL) were added. After the mixture was heated at reflux for 30 minutes, the solvent was removed in vacuo. The residue was purified by preparative HPLC (RT=49.9 minutes, YMC-Triart C18, 20 mmID×250 mm, linear gradient; with 0 to 100% acetonitrile in 0.1% aqueous TFA solution at a flow rate of 8 mL/min for 40 minutes). A red solid compound Q (15.0 mg, 18%) was obtained.

¹H NMR (500 MHz, CDCl₃): δ 8.64 (s, 1H), 7.54 (dd, J=10.3, 4.6 Hz, 1H), 7.42 (d, J=9.2 Hz, 1H), 6.95 (d, J=15.5 Hz, 1H), 6.82-6.87 (m, 2H), 6.85 (d, J=8.6 Hz, 2H), 3.04 (s, 3H)

¹³C{¹H}NMR (126 MHz, CDCl₃): δ 172.1, 158.0, 152.3, 148.3, 144.8, 143.6, 142.9, 134.9, 128.32, 128.27, 124.1, 123.1, 122.1, 121.6, 110.8, 51.6, 39.2, 28.2, 22.4

HRMS (ESI⁺):m/z calcd for C₁₉H₁₇N₄SBrNa⁺[M+Na]⁺435.0255. Found 435.0251.

### 1-3c. Synthesis of Compound J

A compound J was synthesized by Knoevenagel condensation of a julolidine derivative and the compound 5 as shown in the following synthesis scheme.

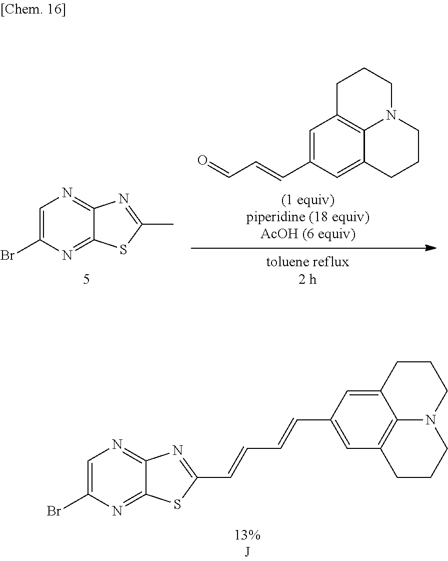 [SMILES: O=C/C=C/c1cc2c3c(c1)CCCN3CCC2 ; Cc1nc2ncc(Br)nc2s1 ; Brc1cnc2nc(/C=C/C=C/c3cc4c5c(c3)CCCN5CCC4)sc2n1]

### Synthesis of Compound J (6-Bromo-2-((1E,3E)-4-(2,3,6,7-tetrahydro-1H,5H-pyrido[3,2,1-ij]quinolin-9-yl)butadien-1,3-dien-1-yl)thiazolo[4,5-b]pyrazine)

To a mixture of the compound 5 (4.8 mg, 0.02 mmol) and Ald2 (4.3 mg, 0.02 mmol) in toluene (2 mL), AcOH (12.0 μL, 0.12 mmol) and piperidine (20.7 μL, 0.36 mmol) were added. After the mixture was heated and refluxed for 2 hours, the solvent was removed in vacuo. The residue was purified by preparative HPLC (RT=43.2 minutes, YMC-Triart C18, 20 mmID×250 mm, linear gradient; with 0 to 100% acetonitrile in 0.1% aqueous TFA solution at a flow rate of 8 mL/min for 40 minutes). A red solid compound J (1.1 mg, 13%) was obtained.

¹H NMR (CDCl₃): δ 8.61 (s, 1H), 7.50 (dd, J=14.9, 4.6 Hz, 1H), 6.97 (s, 2H), 6.84 (d, J=15.5 Hz, 1H), 6.74-6.79 (m, 2H), 3.23 (t, J=5.2 Hz, 4H), 2.75 (t, J=6.3 Hz, 4H), 1.94-1.99 (m, 4H)

¹³C{¹H}NMR (CDCl₃): δ 171.7, 157.6, 151.8, 144.2, 143.3, 142.6, 134.3, 126.8, 122.8, 121.2, 121.2, 120.7, 49.9, 27.6, 21.5

HRMS (ESI⁺): m/z calcd for C21H₁₉N₄SBrNa⁺[M+Na]⁺461.0411 found 461.0400

### 1.4 Synthesis of Compounds C2OH, O, and O2

For the purpose of improving cell membrane permeability of the compound Q, the following compounds C2OH, O, and O2 were synthesized as derivatives in which a substituent having a hydroxyl group was introduced into an aniline site while maintaining the skeleton of the compound Q.

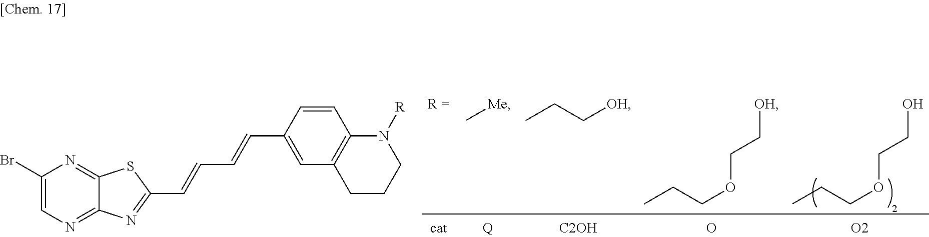

### Synthesis of Compound C2OH (2-(6-((1E,3E)-4-(6-bromothiazolo[4,5-b]pyrazin-2-yl)butadien-1,3-dien-1-yl)-3,4-dihydroquinolin-1(2H)-yl)ethane-1-ol)

A compound C2OH was synthesized according to the following scheme. Specifically, S_{N2} reaction to 2-bromoethyl acetate with 1,2,3,4-tetrahydroquinoline, Vilsmeir reaction, and a deprotection reaction of an acetyl group at the end were performed, and purification was finally performed to obtain a compound 8 at a three-step yield of 48%. Subsequently, a compound 9 was obtained by Horner-Wadsworth-Emmons reaction and further deprotection. Finally, Knoevenagel condensation of the compound 5 and the compound 9 was performed to obtain a red solid compound C2OH (12.3 mg, 13.3%).

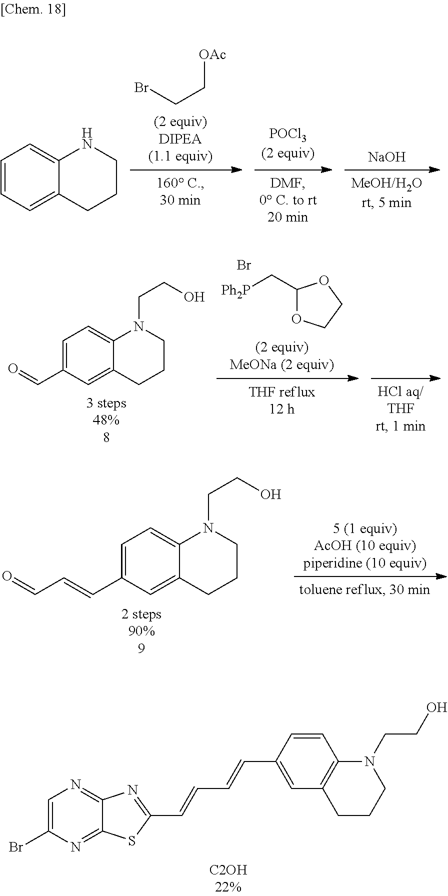

### Synthesis of Compounds O and O2

Further, two types of the compound O (n=1) and the compound O2 (n=2) in which an ether linker was introduced between a tetrahydroquinoline site and a hydroxy group were synthesized by the following synthesis scheme.

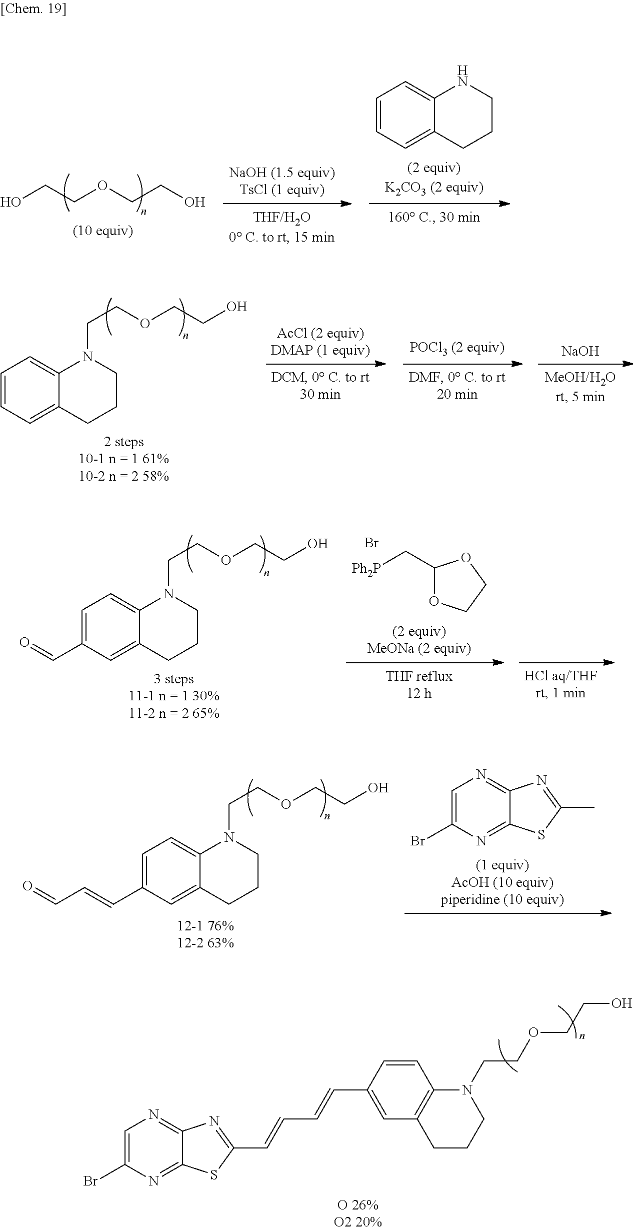

### 2. Verification of Absorption Wavelength

It can be said that the longer the wavelength of light used for a living body, the lower the cytotoxicity and the better the tissue permeability. Therefore, the UV-vis spectrum of the compound of the present invention was observed to examine whether excitation was possible by light irradiation with less cytotoxicity. As a result, the observed maximum absorption wavelength (λₘₐₓ) was as shown in the following table.

TABLE 1

| Compound | P | PzT | OMe | Q | J |
|---|---|---|---|---|---|
| λₘₐₓ (nm) | 437 | 468 | 495 | 500 | 508 |

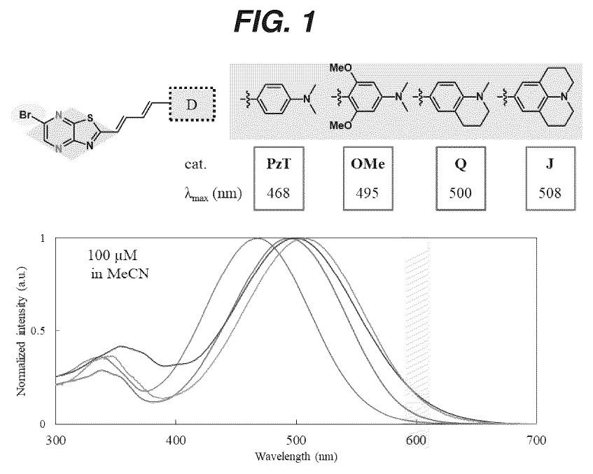

All the compounds had an absorption band in a region of 540 nm or more where cytotoxicity was relatively low. In particular, it was found that the compounds OMe, Q, and J had an absorber on the long wavelength side of 595 nm or more where cytotoxicity can be ignored in an acetonitrile solvent (FIG. 1).

### 3. Intracellular Photooxygenation Reaction

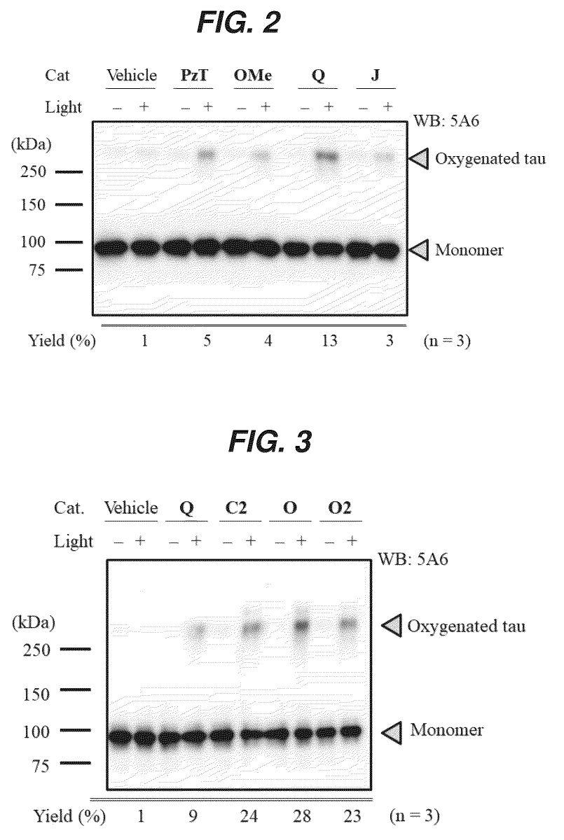

An intracellular oxygenation reaction by the compounds PzT, OMe, Q, and J of the present invention was performed using HEK cells in which aggregated tau was overexpressed. Specifically, 10 uM of the compound of the present invention was added to a cell medium, and then irradiated with light at 595 nm for 15 minutes. After the reaction, viable cells were collected and lysed, and then the intracellular photooxygenation reaction was evaluated by measurement with Western blotting (FIG. 2). In a case where the compound of the present invention was not added, tau was detected between 75 kDa and 100 kDa by the 5A6 antibody that is a tau specific antibody, and a theoretical molecular weight of the overexpressed tau was about 70 kDa, and therefore, the tau was identified as a monomer. On the other hand, in a case where the compound of the present invention was used, a crosslinked product, which is one of products of the photooxygenation reaction, was detected in a region (more than 250 kDa) suggesting a molecular weight 4 times that of the monomer.

Similarly, the compounds of the present invention C2OH, O, and O2 were also subjected to an intracellular oxygenation reaction. The cells overexpressing aggregated tau were incubated with a medium containing 1 μM of the compound of the present invention for 10 minutes. Thereafter, light of 595 nm was applied for 15 minutes. Reaction progress was identified and quantified by Western blotting measurements (FIG. 3). Note that since these compounds have higher solubility in water than the compound Q, the amount of these compounds added to the medium was set to 1/10. As a result, it was confirmed that a crosslinked product, which is one of products of a photooxygenation reaction, was detected in a region of more than 250 kDa on the right side of the lane to which the compound was added, whereas no crosslinked body was provided under non-light irradiation conditions, that is, on the left side of each lane in all the compounds, and the reaction proceeded in a light-dependent manner. The compounds C2OH, O, and O2 into which a hydroxyl group was introduced were twice or more as active as the compound Q.

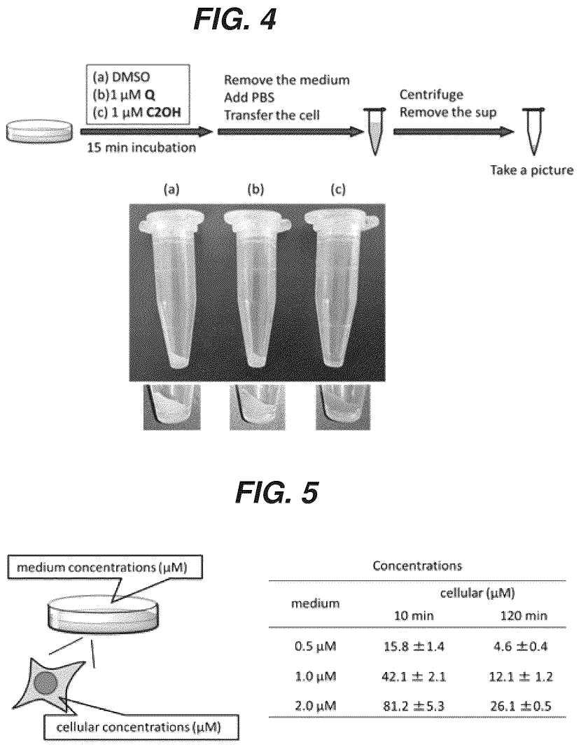

The reason why the three compounds C2OH, O, and O2 showed higher activity than the compound Q is considered that introduction of a hydroxy group was effective for cell membrane permeation. When the cells were recovered after incubating the cultured cells (Fs17) with a medium containing 1 μM of the compound Q and the compound C2OH having a hydroxy group for 15 minutes, the pellets sprinkled with the compound C2OH was colored with a color derived from the compound of the present invention, and it was suggested that the hydroxy group greatly improved the membrane permeability as compared with the result obtained by the compound A having no hydroxy group (FIG. 4).

Here, the membrane permeability of the compound O showing the most excellent activity in the photooxygenation reaction was quantified. The volume of Fs17 used in the above experiment was approximated to the size of HEK cells as host cells, that is, 1.23 μL, and the total number of cells and the total amount of the compound O contained in the total cells were quantified to determine an average intracellular concentration. The cells were incubated with a medium containing the compound O for 10 minutes, the medium was discarded, and the remaining adherent cells were washed 3 times with PBS. After collecting the adherent cells, the total number was calculated. Subsequently, the cell membrane was broken and centrifuged to obtain pellets containing the compound O. Acetonitrile was added thereto, and the extract was subjected to analytical HPLC to determine a concentration of the compound of the present invention. The result was used to calculate the intracellular concentration (FIG. 5). It was suggested that the intracellular concentration was about 30 times higher than the medium concentration, and it was found to have extremely excellent cell membrane properties. On the other hand, when the incubation time was extended from 10 minutes to 2 hours, it was found that the intracellular concentration was about 1/4. That is, it was suggested that the compound O undergoes metabolism when left for a long time after being sprinkled.

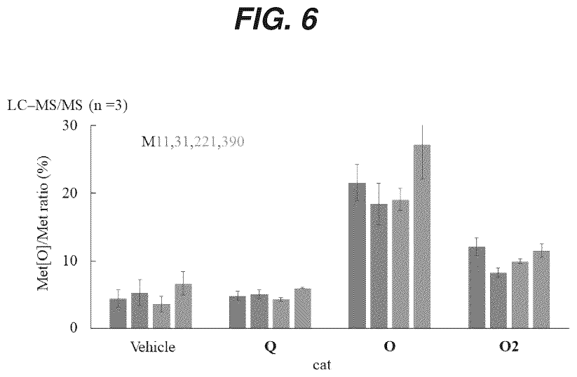

LC-MS/MS analysis was performed on the reaction product in order to confirm the production of side chain oxygenates that were difficult to specifically detect as a result of overlapping with unreacted substances in Western blotting. Since side chain oxygenates are mainly produced by oxidation of methionine side chains to sulfoxide, it was focused on 4 methionines contained in overexpressed tau having a total length of 412 amino acids. The intracellular protein after the reaction was separated according to the molecular weight by SDS-page, monomeric tau was cut out by trypsin enzyme digestion, and LC-MS/MS measurement was performed. A ratio of the detection intensity of the methionine oxidant was calculated from the sum of the detection intensities of the methionine reductant and the oxidant obtained from MS1 in the LC-MS/MS spectrum (FIG. 6). It was clarified that, as compared with the control (Vehicle) conditions, the reaction using the compound Q did not change the oxygenation level of methionine, whereas in a case where the compound O found to exhibit higher activity was used, methionine was oxygenated with a high yield. This result was consistent with a hierarchy of catalytic activities based on the oxygenation yield calculated from Western blotting, and it was supported that the compound O was the most active.

### 4. Identification of Oxygenation Site

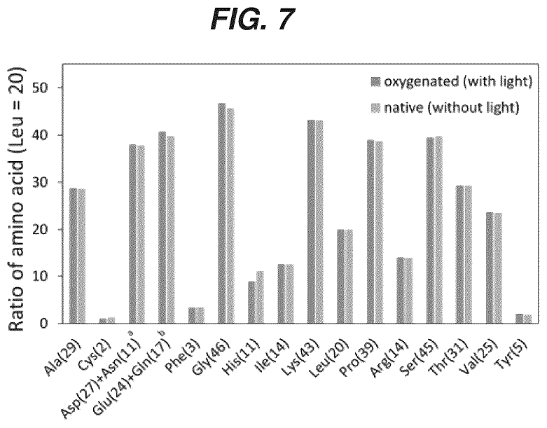

The photooxygenation reaction produces oxygenated tau with oxygenated amino acids. Therefore, by comparing the residual amount of the natural amino acid contained in the oxygenated tau and the unreacted tau by amino acid analysis, it was clarified that the site susceptible to oxygenation was at the amino acid level. After pellets containing oxygenated tau was obtained, the pellets were redissolved in PBS to perform amino acid analysis. In the hydrolysis with acid, the amino acid residual amount with the control condition was compared for each amino acid. Since a peak corresponding to methionine overlaps with a peak of an unidentified impurity and accurate quantification was not obtained, comparison was performed only with other amino acids except methionine (FIG. 7). There was no change in the abundance of arginine, tyrosine, and the like even after the reaction, whereas there was a difference in the residual amount of histidine due to the oxygenation reaction, and it was clarified that histidine is a main oxygenation site. In addition, there was a slight difference in the abundance of cysteine residues, which suggested that the cysteine residues were reacted by oxygenation. Histidine was found to be the main oxygenation site.

Next, the pellets containing oxygenated tau were redissolved with an aqueous ammonium hydrogen carbonate solution, which is a solvent that can be used for LC-MS/MS. After urea (final concentration 8M) was added for disaggregation, enzymatic digestion (trypsin for 12 hours or Asp-N/trypsin for 12 hours/4 hours) was performed. The solution was subjected to LC-MS/MS measurement, and the ionic strength of the peak derived from a fragmented peptide containing histidine was compared between the two conditions (oxygenation reaction and control) to calculate a residual ratio of each fragment after the photooxygenation reaction. However, H300 and H301 were not be detected. When the residual ratios calculated as described above were compared, it was clarified that clear that H239 and H270 were greatly reduced. Since the residual ratio should theoretically be 1 when no reaction occurs, it indicates that the histidine in these two H239 and H270 is converted into another structure. As an amino acid sequence that plays a central role in aggregation of tau, ²⁴⁶VQIINK²⁵¹ and ²⁷⁷VQIVYK²⁸² have been reported, but considering that these aggregation cores are present in the vicinity of H239 and H270 and are central sites of sites (MBRD regions) forming a cross R sheet of aggregated tau, it can be said that the results support that the compound O causes the reaction to proceed in an aggregate-selective manner. From the above, it has been clarified that the photooxygenation reaction to tau is a reaction in which mainly H239 and H270 undergo oxygenation.

### 5. Validation of Cytotoxicity

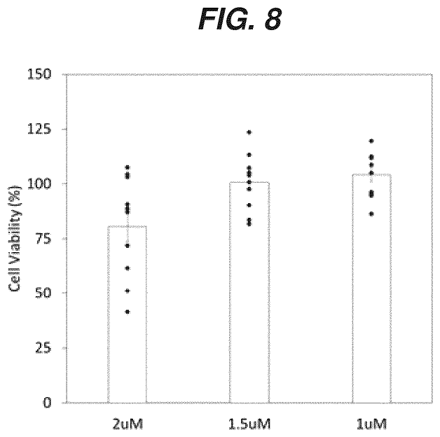

Although the compound of the present invention causes dramatic cell death when singlet oxygen is non-specifically produced in cells, it has been clarified that the compound of the present invention can perform a photooxygenation reaction avoiding cell death. Various concentrations (2.0 μM, 1.5 μM, and 1.0 μM) of the compound O were added to the cell culture medium, and after incubation with Fs17 cells for 10 minutes, the culture medium was replaced and irradiated with 595 nm light for 15 minutes. Thereafter, the medium was replaced with a medium not containing the compound of the present invention, and the cells were incubated for 24 hours to measure cell viability (FIG. 8). As a result, in the photooxygenation reaction in which 2 μM was added to the medium, the cell viability was about 80%, but cell death was not observed under the addition condition of 1.5 μM or less. It was found that the compound O does not non-specifically produce singlet oxygen within an appropriate concentration range in the cells.

### 6. Photooxidation Reaction in Model Mouse (in Vivo)

The photooxygenation reaction of the compound O of the present invention in vivo was evaluated using a model mouse.

### 6-1 Evaluation of Blood-Brain Barrier (BBB) Permeability and Photooxygenation Reaction in Brain

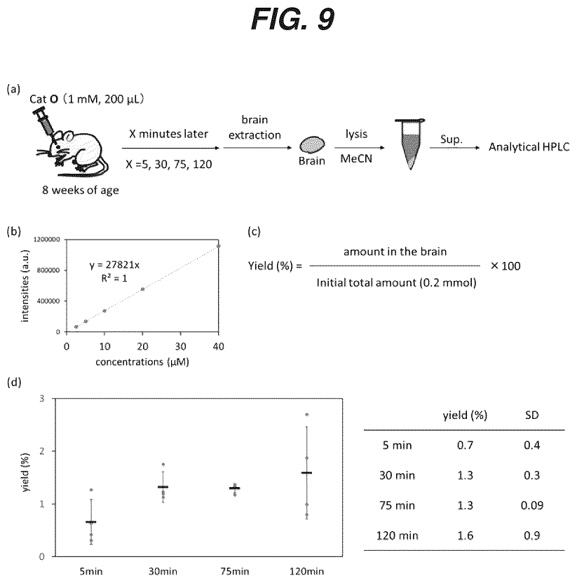

First, the blood-brain barrier (BBB) permeability of the compound of the present invention in a mouse was evaluated. A wild-type model mouse (8-week-old) was injected with 1 mM of the compound O in an amount of 200 μL from under the eyes, after waiting for each time (5, 30, 75, and 120 minutes), the brains were extracted and lysated with acetonitrile, the supernatant was measured by analytical HPLC to calculate the content of the compound O, and the permeation yield was determined from the ratio to the dose (FIG. 9). After 5 minutes from the administration, the compound O started to migrate into the brain, and after 30 minutes from the administration, the yield exceeded 1%. After 120 minutes, a yield improvement of about 0.3% was observed, but large variations were included. On the other hand, under the conditions after 5 minutes and after 30 minutes, it was visually observed that a characteristic red color was mixed in the compound O in the periphery and blood, but the color of the compound of the present invention disappeared after 60 minutes from the injection. Since there is a concern that the compound remaining at a site other than the brain may lead to side reactions, the optimal condition was to start the reaction 75 minutes after the injection in order to promote the metabolism in the blood and the periphery as much as possible.

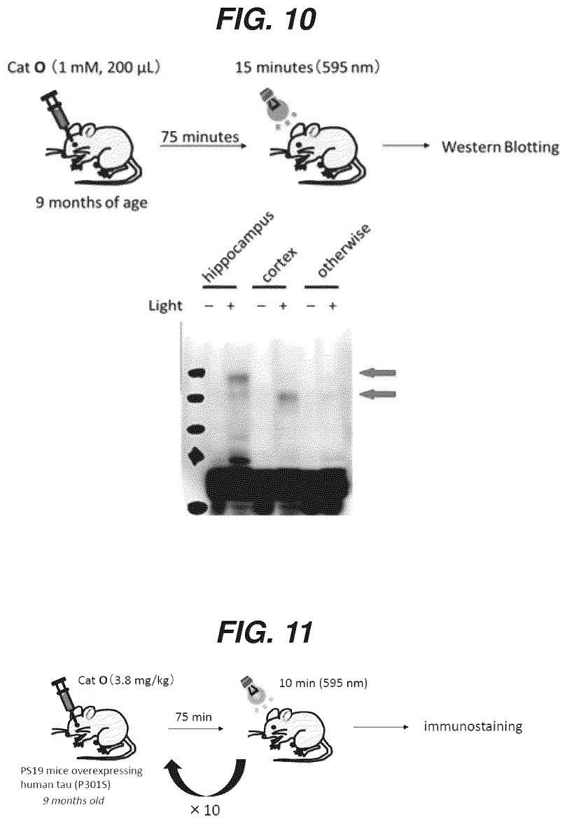

Next, an intracellular photooxygenation reaction in the mouse brain was performed. A 9-month-old model mouse in which aggregated tau was accumulated was injected with the compound O from under the eyes, and 75 minutes after that, the mouse was irradiated with light for 15 minutes. Thereafter, the brain was immediately extracted, the brain was divided into the hippocampus, the cortex, and others, and the response was analyzed by Western blotting. Under the condition with light, that is, on the right of each lane, crosslinked product bands were observed in the hippocampus and the cortex, and the progress of the photooxygenation reaction was confirmed (FIG. 10). This result demonstrates that tau amyloids in the brain can be photooxygenated in vivo in a non-invasive manner by using the compound of the present invention.

Note that, considering that the aggregation of tau starts from the hippocampus and that the cortex, which is the outermost surface of the brain, is likely to receive external irradiation light, the hippocampus and the cortex are sites having an advantageous element for the photooxygenation reaction. Therefore, the present result of forming a crosslinked product in the hippocampus preferentially over other sites further supports the progress of the photooxygenation reaction.

### 6-2 Comparison of Neuronal Cell Death Suppressing Effect

Furthermore, the effect of suppressing reduction of tau and neuronal cell death in the mouse brain by photooxygenation was compared and evaluated. A 9-month-old tau transgenic mouse (PS19 mouse) was administered with the compound O at 3.8 mg/kg through the supraorbital vein, and 75 minutes later, the mouse was irradiated with light of 595 nm for a total of 10 minutes. The procedure was repeated once a day for a total of 10 times, and then the brain was collected and subjected to immunohistochemical studies using a phosphorylated tau antibody (AT8) and an antibody against a neuronal marker NeuN (FIG. 11). Here, AT8 is an antibody used for detection of tau accumulation pathology, and NeuN is generally a neuronal cell marker.

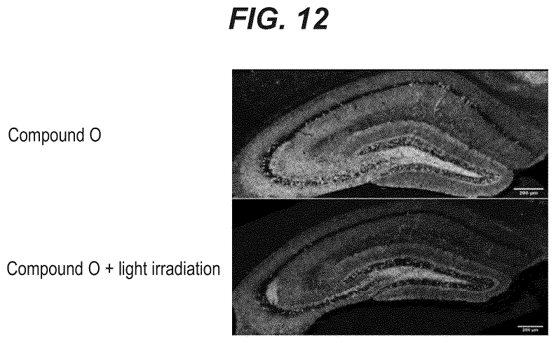

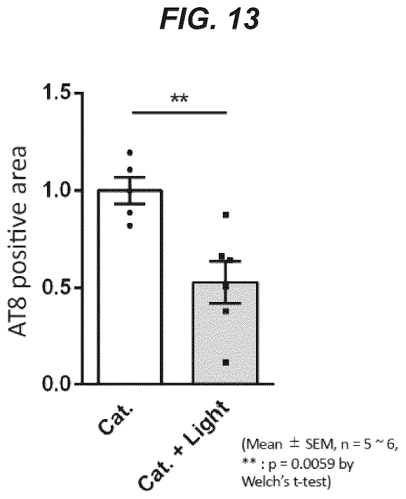

As a result, it was found that the AT8 positive region was significantly reduced by administering the compound O and irradiating with light (FIGS. 12 and 13). This result indicates that the tau accumulation pathology was reduced by photooxygenation.

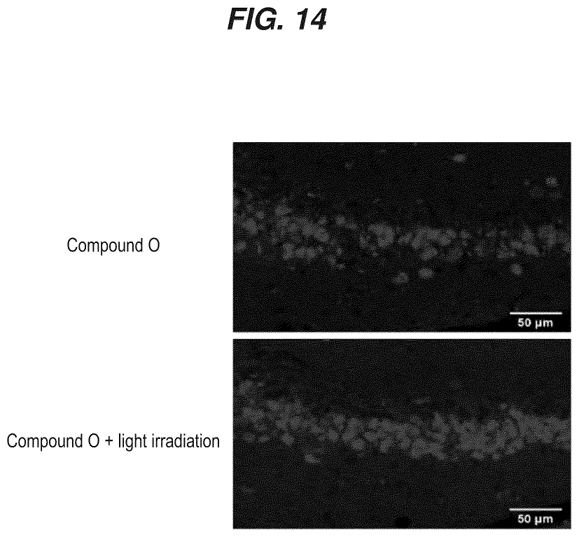

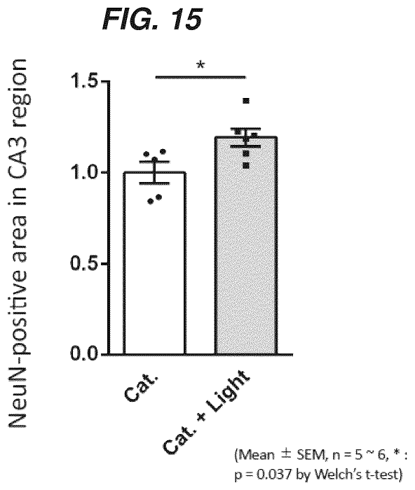

On the other hand, it was found that administration of the compound O and light irradiation increased the number of neurons in hippocampal CA3 (FIGS. 14 and 15)

1. A compound represented by the following Formula (I) or a salt thereof: 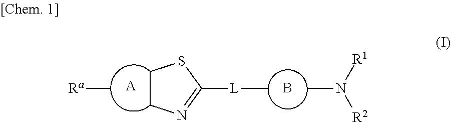
(wherein a ring A is an aromatic ring which may be substituted or a heteroaromatic ring which may be substituted; a ring B is an aromatic ring which may be substituted or a heteroaromatic ring which may be substituted; L is a conjugate spacer; R^{a} is a halogen atom or a haloalkyl group having 1 to 3 carbon atoms, and may be present at any position on the ring A; R¹ and R² may be independently the same or different, and each are independently a hydrogen atom or a substituent selected from the group consisting of an alkyl group, an alkoxy group, a hydroxyalkyl group, an ether group, a hydroxyether group, an aminoalkyl group, a thioalkyl group, and a thioether group which may be substituted, and in a case where R¹ and/or R² is an alkyl group, R¹ and/or R² may form a ring structure containing a nitrogen atom to which R¹ and/or R² is bonded and optionally an atom constituting the ring B; and the N atom to which R¹ and R² are bonded may be present at any position on the ring B).

2. The compound according to claim 1, wherein the ring A is a 4- to 6-membered monocyclic aromatic ring or heteroaromatic ring.

3. The compound according to claim 1 or 2, wherein the ring B is a 4- to 6-membered monocyclic aromatic ring or heteroaromatic ring, or an 8- to 12-membered bicyclic aromatic ring or heteroaromatic ring.

4. The compound according to any one of claims 1 to 3, wherein R^{a} is Br or I.

5. The compound according to any one of claims 1 to 4, wherein L is an alkenylene group having a conjugated double bond, an aryl group, or a combination thereof.

6. The compound according to any one of claims 1 to 5, wherein R¹ and R² may be independently the same or different and each are a linear or branched alkyl group having 1 to 10 carbon atoms, a linear or branched hydroxyalkyl group having 1 to 10 carbon atoms, or a linear or branched hydroxyether group having 1 to 10 carbon atoms.

7. The compound or the salt thereof according to claim 1, wherein the compound or the salt thereof is represented by the following Formula (II): 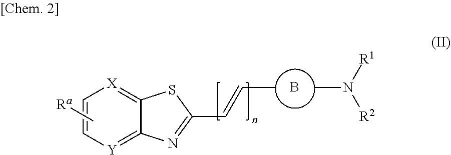
(wherein the ring B, R^{a}, R¹, and R² are as defined in claim 1; X and Y may be independently the same or different, and each are a carbon atom or a nitrogen atom; and n is a natural number of 1 to 5).

8. The compound according to claim 7, wherein X is a nitrogen atom, and Y is a carbon atom or a nitrogen atom.

9. The compound or the salt thereof according to claim 1, wherein the compound or the salt thereof is represented by the following Formula (III): 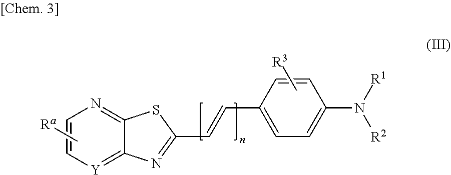
(wherein R^{a}, R¹, and R² are as defined in claim 1; Y is a carbon atom or a nitrogen atom; R³s may be independently the same or different, and each are a hydrogen atom or 1 to 4 substituents selected from arbitrary substituents; and n is a natural number of 1 to 5).

10. The compound according to claim 9, wherein one or two of R³s are alkoxy groups.

11. The compound or the salt thereof according to claim 9, wherein the compound or the salt thereof is represented by any one of the following Formulas (III-a) to (III-c): 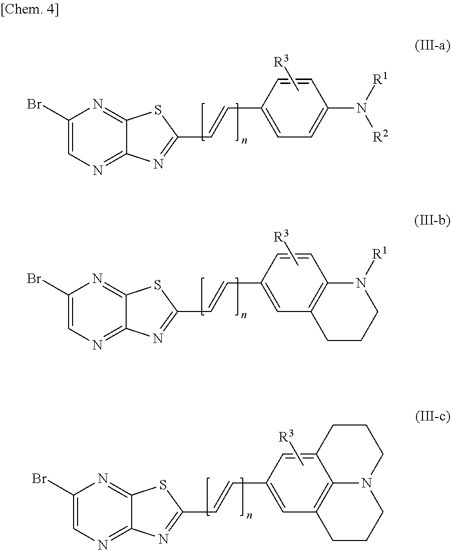
(wherein R¹, R², R³, and n are as defined in claim 9).

12. A photooxygenation catalyst for pathogenic amyloids, comprising the compound or the salt thereof according to any one of claims 1 to 11.

13. An aggregation inhibitor for pathogenic amyloids, comprising the compound or the salt thereof according to any one of claims 1 to 11.

14. The photooxygenation catalyst according to claim 12 or the aggregation inhibitor according to claim 13, wherein the pathogenic amyloids are tau proteins.

15. A pharmaceutical composition comprising the compound or the salt thereof according to any one of claims 1 to 11 and a pharmaceutically acceptable carrier.

16. The pharmaceutical composition according to claim 15, wherein the pharmaceutical composition is an agent for preventing or treating a disease associated with pathogenic amyloids.

17. The pharmaceutical composition according to claim 16, wherein the disease associated with pathogenic amyloids is Alzheimer's disease.

18. The pharmaceutical composition according to any one of claims 15 to 17, wherein the pathogenic amyloids are tau proteins.

19. A use of the compound or the salt thereof according to any one of claims 1 to 11 for preparing an agent for preventing or treating a disease associated with pathogenic amyloids.

20. A method for preventing or treating a disease associated with pathogenic amyloids, the method comprising administering an effective amount of the compound or the salt thereof according to any one of claims 1 to 11.

21. The method according to claim 20, further comprising, after the administering of the compound or the salt thereof according to any one of claims 1 to 11, irradiating an affected area of a patient with light from outside a body.

22. The method according to claim 20 or 21, wherein the disease associated with pathogenic amyloids is Alzheimer's disease.

23. The method according to any one of claims 20 to 22, wherein the pathogenic amyloids are tau proteins.
